(19) United States
(12) Patent Application Publication
(10) Pub. No.: US 2017/0313987 A1
(43) Pub. Date: Nov. 2, 2017

(54) METHOD OF MANUFACTURING PANCREAS ISLET OF LANGERHANS MIMICS USING INDUCED PLURIPOTENT HUMAN STEM CELLS

(71) Applicant: StemoniX Inc., Eden Prairie, MN (US)

(72) Inventor: Robert John Petcavich, The Woodlands, TX (US)

(21) Appl. No.: 15/581,502

(22) Filed: Apr. 28, 2017

Related U.S. Application Data
(60) Provisional application No. 62/329,288, filed on Apr. 29, 2016.

(51) Int. Cl. C12N 5/071; A61K 35/39; C12N 5/00

(52) U.S. Cl. CPC C12N 5/0677; C12N 5/0012; A61K 35/39; C12N 2506/45; C12N 2533/54; C12N 2535/00; C12N 2525/00

(57) ABSTRACT

The present disclosure provides a method of manufacturing pancreas Islet of Langerhans (IOL) mimetics using induced human pluripotent stem cells (iPSc) and porous micro carrier scaffolds that allow for subsequent vascularization and/or innervation.

### CROSS-REFERENCE TO RELATED APPLICATIONS

This application claims the benefit of the filing date of U.S. application Ser. No. 62/329,288, filed on Apr. 29, 2016, the disclosure of which is incorporated by reference herein.

### BACKGROUND

Diabetes mellitus type 1 (also known as type 1 diabetes, or T1D; formerly insulin-dependent diabetes or juvenile diabetes) is a form of diabetes mellitus that results from the autoimmune destruction of the insulin-producing beta cells in the pancreas. The subsequent lack of insulin leads to increased glucose in blood and urine. The classical symptoms arepolyuria (frequent urination), polydipsia (increased thirst), polyphagia (increased hunger) and weight loss.

The cause of diabetes mellitus type 1 is unknown. Type 1 diabetes can be distinguished from type 2 by autoantibody testing. The C-peptide assay, which measures endogenous insulin production, can also be used.

Administration of insulin is essential for survival. Insulin therapy must be continued indefinitely and typically does not impair normal daily activities. People are usually trained to independently manage their diabetes; however, for some this can be challenging. Untreated, diabetes can cause many complications. Acute complications include diabetic ketoacidosis and nonketotic hyperosmolar coma. Serious long-term complications related to high blood sugar include heart disease, stroke, kidney failure, foot ulcers and damage to the eyes. Furthermore, complications may arise from low blood sugar caused by excessive insulin treatment.

Diabetes mellitus (DM) type 1 accounts for between 5% and 10% of all diabetes cases. Globally, the number of people with DM type 1 is unknown although it is estimated that about 80,000 children develop the disease each year. Within the United States the number of affected persons is estimated at one to three million. The development of new cases vary by country and region; the lowest rates appear to be in Japan and China with approximately 1 person per 100,000 per year; the highest rates are found in Scandinavia where it is closer to 35 new cases per 100,000 per year. The United States and other countries in northern Europe fall somewhere in between with 8-17 new cases per 100,000 per year.

Insulin is produced by the beta cells within the pancreas, which are contained within ball-like structures called the islets of Langerhans (IOL). Beta cells (β-cells) are highly sensitive to changes in blood glucose and secrete active (fully processed) insulin in response to elevated blood glucose. Islets also contain other cell types that produce other hormones, such as glucagon (produced by alpha cells). The beta cells are held tightly in a 3-dimensional ball, surrounded by alpha cells (α-cells).

The three-dimensional structure of a pancreatic islet ensures that there is direct cell-cell contact enabling beta cells to communicate with their neighboring cells. Such structure and communication is vital for the exquisite control of blood glucose required to maintain a healthy functioning organism. Not only are the cells packed together in an islet, the islet itself is held within the structure of the pancreas. There are approximately 3,000,000 Islets of Langerhans in a typical human pancreas with a combined weight of approximately 2 grams.

Scientific efforts have concentrated on trying to develop sources of insulin-producing cells (e.g., from stem cells) and looking at ways of delivering them into recipients.

Even if it was possible to produce an insulin-producing cell from a stem cell, the problem still remains as to how to introduce that cell into a recipient. Many different avenues for delivery has been explored around the world, most focusing on some kind of encapsulation in an artificial substrate such as alginates or polymers. To date there has been no truly successful method of encapsulation. Encapsulation is limited by the need to ensure oxygen and nutrient diffusion to cells as well as allowing insulin to leave the cells. To date the most promising approach to encapsulation and implantation has been disclosed by Viacyte Inc. of San Diego, Calif.

### SUMMARY

The present disclosure provides insulin-producing Islet of Langerhans (IOL) mimics prepared from stem cells such as iPSc and a microgravity cell culture manufacturing method environment in combination with an enzyme(s) and biodegradable micro carrier scaffolds. Together, these allow the generation of transplantable 3D insulin-producing IOL mimics which function like normal islets, and which can be transplanted into patients using the same transplant techniques currently being employed to deliver donated islets, and/or which provide a more patient compatible transplantation of IOL's manufactured from, for example, the patient's own skin cells using iPSc as the source to prevent immune rejection.

In one embodiment, a method of manufacturing cells for use in a graft in vivo or in a transplant in a mammalian organ is provided by the use of stem cells, e.g., iPSc, porous micro carrier scaffolds, a microgravity reactor, and one or more proteolytic enzymes. In one embodiment, a method of manufacturing IOL pancreas mimics is provided by the use of iPSc pancreas progenitor cells, porous micro carrier scaffolds, a microgravity reactor, and one or more proteolytic enzymes.

In one embodiment, a method of manufacturing IOL mimics is provided. The method includes culturing, e.g., in liquid media, pancreatic cells or pancreatic progenitor cells and biocompatible microporous particles formed of a biodegradable material, which particles comprise pores leading to microchannels, under conditions that allow for expansion of the cells and optionally differentiation of the progenitor cells. In one embodiment, the conditions allow for coating of the pancreatic cells into and on the particles and the pores. In one embodiment, the conditions allow for coating of the pancreatic progenitor cells into and on the particles and the pores and differentiation of those progenitor cells.

After the cells adhere or attach and proliferate, the particles are removed, leaving cells in a three dimensional matrix having microchannels. In one embodiment, the particles are degraded without substantially affecting the viability or biological activity of the cells. In one embodiment, the culturing occurs in a microgravity reactor. In one embodiment, the cells are pancreatic progenitor cells and the conditions optionally include factors that provide for differentiation to alpha cells, beta cells, delta cells, or any combination thereof. In one embodiment, the cells are obtained from iPSc. In one embodiment, the particles are degradable by one or more enzymes. In one embodiment, the particles comprise gelatin. In one embodiment, the particles are degradable by trypsin, cellulase, dextranase, gelatinase, pepsin, pancreatin, papain, or bromelain, or any combination thereof. In one embodiment, the particles have an average diameter of about 100 microns to about 250 microns. In one embodiment, the particles have an average diameter of about 50 microns to about 200 microns. In one embodiment, the particles have an average diameter of about 50 microns to about 250 microns. In one embodiment, the particles have an average diameter of about 100 microns to about 200 microns. In one embodiment, the pores in the particles have an average diameter of about 5 microns to about 40 microns. In one embodiment, the pores have an average diameter of about 10 microns to about 30 microns. In one embodiment, the pores have an average diameter of about 8 microns to about 20 microns. In one embodiment, the pores have an average diameter of about 20 microns to about 40 microns. In one embodiment, the pores have an average diameter of about 30 microns to about 40 microns. In one embodiment, the IOL mimic having cells and microchannels formed in a pattern that mimics the pores having microchannels in the particles, is encapsulated in a biocompatible polymer, e.g., a biocompatible film. In one embodiment, the mimic comprises a film having a thickness from about 10 microns to about 1 millimeter. In one embodiment, the film has a thickness of about 100 to about 200 microns. Further provided is an IOL mimic having the cells and microchannels prepared by the method described herein. In one embodiment, the mimic comprises a three dimensional population of cells in the general shape of microparticles having a diameter from about 10 microns to about 1 millimeter. In one embodiment, the mimic comprises a three dimensional population of cells in the general shape of microparticles having a diameter of about 100 to about 200 microns. In one embodiment, the IOL mimic is used to inhibit or treat diabetes, e.g., in a patient having type 1 diabetes.

Therefore, in one embodiment, a IOL mimic is provided that has alpha, beta and gamma cells arranged in a three dimensional structure that enables vascularization when implanted into a living organism, e.g., a mammal such as a bovine, equine, canine, feline, porcine, ovine, caprine or human, either as a stand alone islet mimic or encapsulated in a polymeric containment device, e.g., a porous film bag, tube, or capsules for permanent implantation where the pores int eh containment device are, for instance, pores <20 microns, allow for vascularization into the containment device, to provide for nutrients and removal of waste. The IOL mimic, e.g., having cells derived from the patient, prevents or inhibits immune system rejection and attack. In order to maintain physiological function, beta cells are held together and benefit from good oxygen delivery, nutrient delivery and removal of waste products generated by cell respiration. In vivo, this is ensured by the highly vascularized nature of a pancreatic islet, and in the present disclosure, it is ensured by microchannels, e.g., where some channels connect and others do not in the cell foam-like structure, that remain after the scaffold is removed.

In one embodiment, an IOL mimic is provided that incorporates alpha, beta and gamma cells arranged in a 3 dimensional structure that is derived from mammalian skin cells that are reprogrammed into iPSc and subsequently differentiated into IOL mimics using those progenitor cells, which are contacted with microcarrier scaffolds and cultured in microgravity reactors. After, the cell containing microcarrier scaffolds are contacted with one or more proteolytic enzymes. In order to have a functional IOL mimic, all 3 cell types are present in the Islet 3D configuration and the structure remaining after removal of the scaffold is capable of supporting a vascular or capillary network and optionally a neural network. Without the vascularization, the cells on the interior of the IOL would be subject to hypoxia.

### BRIEF DESCRIPTION OF THE DRAWING

FIG. 1 shows a cross sectional view of a pancreas Islet of Langerhans (IOL) structure. The pancreas has two functions: to make hormones (endocrine) and to produce enzymes for digestion (exocrine). The islets of Langerhans make the hormones insulin and glucagon. The alpha cells 10 make the glucagon and the beta cells 20 produce insulin and the delta cells 30 make somatostatin.

### DETAILED DESCRIPTION

### Bioreactors

The use of bioreactors to produce various human cell and stem cell microstructures has been described.

For example, WO 97/16536, which is incorporated by reference herein, refers to methods for the ex vivo proliferation and differentiation of neonatal and/or adult human or non-human pancreatic islets. The cells are cultured in a microgravity environment and an aggregation medium is employed. The intention is to produce products useful for the treatment of diabetes. It is important to note that WO 97/16536 describes work with adult human tissue and not embryonic stem cells. The tissue used is a donated pancreatic cell from cadavers. The work involves disaggregating the pancreatic islets and then reassembling them using a culture system, and there is a proposal to co-culture with other adult cell types.

In addition, US 2011/0027880, which is incorporated by reference herein, describes the use of a microgravity reactor to produce pancreatic islands. Three-dimensional (3D) insulin-producing cell clusters derived from stem cells (human embryonic stem cells) are provided by the present disclosure, together with a method for their production using a microgravity bioreactor cell culture system.

### Exemplar Methods Using Stem Cells and Microporous Carriers

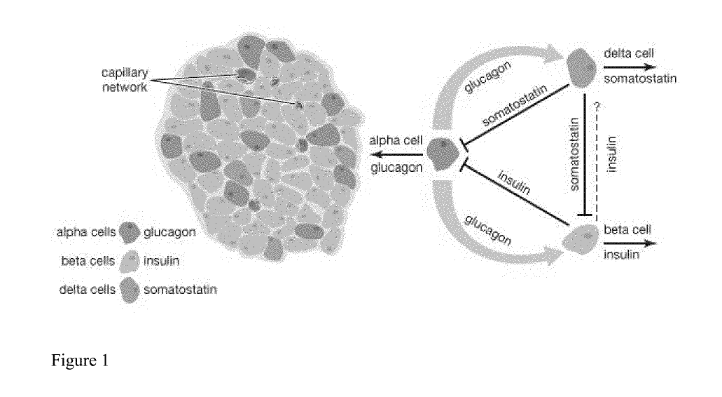

The present disclosure provides for the use of stem cells including iPSc; a microgravity reactor and porous micro carrier scaffolds that are subsequently enzymatically digested for the production of Islets of Langerhans (IOL) mimics that can be subsequently vascularized when implanted into a human patient. FIG. 1 shows a diagram of a cross section containing alpha, beta, and delta cells.

Human pluripotent stem cells (hPSC) have the ability to self renew and proliferate and to differentiate into a wide range of cell types, including pancreas islet cells. Although a number of methods for direct conversion of one differentiated cell type to another have been described, the majority of successful strategies for generating large numbers of differentiated cells attempt to recapitulate the normal development progression to the terminally differentiated state. In these strategies, pluripotent cells are progressively directed by external signals, first to one of the three germ lineages, and then to more specific downstream cell types. The pancreas is derived from the endoderm lineage, which also gives rise to the lung, liver, and gastrointestinal tract.

In one embodiment, inducers of a pathway are used to first generate pancreas progenitor cells, which are then attached to a micro carriers and incubated in a microgravity reactor for about 3 to about 25 days. The micro carriers are then harvested, exposed to an enzyme, such as trypsin, to degrade the micro carrier matrix, which in one embodiment comprises gelatin, to result in a porous IOL mimic that is then optionally stored in growth media until it is ready for implantation.

In one embodiment, the differentiation process starts with human skin cells that are reprogrammed, e.g., by transfection with Sendai virus, to induced pluripotent stem cells (iPSc) by techniques well known in the art. Once the iPSc are generated, the next step is to induce endoderm formation which is generally about a 3 to 7 day process induced by one or more of LY, ActivinA, BMP4, and FGF2 signaling factors. Next gut patterning occurs which again is about a 3 to 5 day process induced by, for example, IWR signaling factor. The next steps involve pancreas specification, proliferation, and progenitor formation that is about a 10 to 20 day process involving growth and signaling factors, such as BMP4, IWr1, SB, HGF, CHIR, vEGF, TGFb, CE, or any combination thereof.

For exemplary factors and conditions for pancreatic lineage differentiation from, for instance, human stem cells, see Shih et al., Ann. Rev. Cell Dev. Biol., 29:81 (2013) the disclosure of which is incorporated by reference herein

### Microcarrier Beads

Macroporous beads, in which anchorage-dependent cells have the possibility to utilize the interior surface, substantially reduce the problems associated with the culture of these cells. Microcarriers have been manufactured from different synthetic materials including dextran, such as GE Biosciences Cytodex, polyacrylamide, polystyrene and cellulose. Cell attachment to these charged microcarriers are mediated by ionic attractions. Cells also attach to gelatin, but through a different mechanism: a protein, fibronectin, has a biospecific binding to gelatin and as the cells has an affinity to this protein they will attach to microcarriers of gelatin, which is susceptible to proteolytic enzymes. Cells may thus be released with almost 100% viability by dissolution of the matrix with an enzyme, e.g., trypsin. Table 1 shows the typical properties of exemplary micro carrier scaffolds that may be employed in the present methods.

TABLE 1

| | | |
|---|---|---|
| Particle diameter (μm) | 130-180 | 130-180 |
| Volume (mL/g dry) | 12-18 | 10-16 |
| Density (g/mL) | 1.04 | 1.04 |
| Microcarriers | 1,000,000 | 800,000 |
| number/gm | | |
| Average Pore diameter | 20 | 20 |
| (μm) | | |

Pancreas progenitor cells are cultured on the beads that are loaded into a microgravity reactor, see, e.g., U.S. Pat. Nos. 5,437,998, 5,155,035 and 5,989,913, the disclosures of which are incorporated by reference herein. Cultures of cells grown on CultiSpher (Percell Biolytia, Astorp Sweden), can be scaled-up in steps of 50 times; for instance, cells harvested from a 1 liter fermentor are sufficient to inoculate a 50 liter fermentor. This is possible due to the macroporous structure and, in one embodiment, a digestible matrix such as a digestible gelatin matrix. The macroporous structure allows the cells to increase from about 10 to 20 cells up to about 2000 to 3000 cells on each bead. As the matrix may be digested with tissue culture grade enzyme, e.g., trypsin, many of not all of these cells can be recovered and used for scale-up purposes.

Used at 1 g/L, cell concentrations of 10×10⁶ cells/mL can be obtained if an efficient oxygen supply system is used. If used in higher concentrations than 2 g/L in standard systems, cells only grow on the outer layer of the microcarriers. Growth is dependent on the oxygen gradient within the system. Cells only grow as long as they are provided with sufficient amounts of oxygen. The pH is usually controlled by carbon dioxide; if a large number of cells are growing inside the beads they will produce an acidic environment. Unless this is controlled it will affect both cell growth and product formation.

The invention will be described by the following non-limiting example.

### Example

A 1 L microgravity reactor is inoculated. CultiSpheres is used at about 2 g/L and contains about 2×10⁶ beads. Each bead is inoculated with at least 10 cells. For example, about 20×10⁶ cells may be used for enhanced attachment and growth. If this concentration is about 0.1×10⁶ cells/mL and the minimum volume that can be used is about 0.5 L, about 50×10⁶ cells are used. The culture may thus be started with about 50×10⁶ cells, which will supply each CultiSphere with about 10 to about 20 cells up to about 2000 to about 3000 cells on each bead. To form the microstructured IOL mimic microchannels for subsequent vascularization, the microcarriers are evenly dispersed. Duplicate samples of about 0.5 mL are taken and after sedimentation of beads, 0.3 mL of the supernatant is removed. Add 0.8 mL trypsin (0.25% w/v in PBS). Mix and incubate at 37° C. for 20 minutes. If the microcarriers have not dissolved after this time, the trypsin concentration is increased. As the matrix is easily digested with tissue culture grade trypsin all the cells can be recovered and used for scale-up and implantation or therapeutic purposes. For example, a protease may be added when the cells have differentiated, e.g., from about 18 to 24 days of starting the culture, and/or insulin secretion is detected. In one embodiment, the protease is contacted with the cell/bead mixture for about 0.2 to 24 hours.

It is to be appreciated that using this process it would be possible to also manufacture other mammalian organ types such as liver, kidney, lung, spleen, brain, eye, skin, nerve, blood vessels, bladders, intestine and heart tissue all of which would benefit from having a porous structure that could be vascularized or innervated to form functional human organs if implanted into a body or used for drug screening and efficacy testing.

The subject matter herein is described by example and different ways of practicing the subject matter have been described. However the subject matter covered by this application is not limited to any one specific embodiment or use or their equivalents. While particular embodiments of the method for fabricating cell micro arrays with subsequent drug dosing have been described it will be appreciated by those skilled in the art that changes and modifications may be made thereto without departing from the invention and as set forth in the following claims.

What is claimed is:

1. A method of manufacturing Islet of Langerhans (IOL) pancreas mimics, comprising:
culturing in liquid media pancreatic cells or pancreatic progenitor cells and biocompatible microporous particles formed of a biodegradable material and having pores, under conditions that allow for coating of the cells into and on the particles and the pores and optionally differentiation of the progenitor cells in and on the particles and the pores; and
degrading the particles under conditions that yield viable pancreatic cells having biological activity, thereby providing for a IOL mimic having a three dimensional population of the pancreatic cells or differentiated progenitor cells interspersed with microchannels.

2. The method of claim 1 wherein the culturing occurs in a microgravity reactor.

3. The method of claim 1 wherein the cells that are cultured are pancreatic progenitor cells and the conditions optionally include factors that provide for differentiation to alpha cells, beta cells, delta cells, or any combination thereof.

4. The method of claim 1 wherein the cells are obtained from iPSc.

5. The method of claim 1 wherein the particles are degradable by one or more enzymes.

6. The method of claim 1 wherein the particles comprise gelatin.

7. The method of claim 1 wherein the particles are degradable by trypsin, cellulase, dextranase, gelatinase, pepsin, pancreatin, papain, or bromelain.

8. The method of claim 1 wherein the particles have an average diameter of about 100 microns to about 250 microns.

9. The method of claim 1 wherein the particles have an average diameter of about 50 microns to about 200 microns.

10. The method of claim 1 wherein the pores have an average diameter of about 5 microns to about 40 microns.

11. The method of claim 1 wherein the pores have an average diameter of about 10 microns to about 30 microns.

12. The method of claim 1 further comprising encapsulating the IOL mimic having the cells and microchannels in a biocompatible polymer.

13. The method of claim 1 wherein about 1×10⁶ to about 5×10⁶ microparticles per liter of media are cultured with the cells.

14. The method of claim 1 wherein about 20×10⁶ to about 100×10⁶ cells per liter of media are cultured with the microparticles.

15. An implantable device comprising an IOL mimic having the cells and microchannels prepared by the method of claim 1.

16. The device of claim 15 wherein the cells comprise a three dimensional population of cells having a diameter of about 10 microns to about 1 millimeter.

17. The device of claim 15 wherein the cells comprise a three dimensional population of having a diameter of about 100 microns to about 200 microns.

18. The device of claim 15 wherein the IOL is encapsulated in a film having a thickness of about 100 microns to about 200 microns.

19. The device of claim 15 which has about 5×10⁶ to 15×10⁶ cells.

20. A method to inhibit or treat diabetes, comprising: administering to a patient in need thereof the device of claim 15.
